(19) United States
(12) Patent Application Publication
(10) Pub. No.: US 2026/0184799 A1
(43) Pub. Date: Jul. 2, 2026

(54) Antibodies that Bind to Colony Stimulating Factor 1 Receptor (CSF-1R) and Uses Thereof

(71) Applicant: Incyte Corporation, Wilmington, DE (US)

(72) Inventors: Cory M. Sutherland, Everett, WA (US); Gilbert Martinez, Lynnwood, WA (US); Cole M. Hauer, Edmonds, WA (US)

(21) Appl. No.: 19/434,771

(22) Filed: Dec. 29, 2025

Related U.S. Application Data
(60) Provisional application No. 63/740,017, filed on Dec. 30, 2024.

(51) Int. Cl. C07K 16/28; A61K 39/00; A61P 37/06

(52) U.S. Cl. CPC C07K 16/2866; A61P 37/06; A61K 2039/505; C07K 2317/92

(57) ABSTRACT

The present disclosure provides antibodies that binds to human colony stimulating factor 1 receptor (CSF-1R) and methods of treating chronic graft-versus-host disease (cGVHD) with the antibodies.

### CROSS-REFERENCE TO RELATED APPLICATION

This application claims the benefit of priority to U.S. Provisional Patent Application No. 63/740,017, filed on Dec. 30, 2024, the entire content of which is hereby incorporated by reference in its entirety.

### SEQUENCE LISTING

This application contains a Sequence Listing that has been submitted electronically as an XML file named “20443-0868001_SL_ST26.XML.” The XML file, created on Dec. 2, 2025, is 16,618 bytes in size. The material in the XML file is hereby incorporated by reference in its entirety.

### BACKGROUND

Chronic graft-versus-host disease (cGVHD) is a severe complication of allogeneic hematopoietic cell transplantation (HCT) that affects various organs leading to a reduced quality of life. cGVHD occurs in up to 50% of allogeneic HCT cases, where donor T- and B-cells derived from the graft recognize and attack host antigens (Socié et al., 2014, Blood, 124:374-84). cGVHD has increased during the last two decades due to increasing patient age and increasing use of unrelated and/or mismatched donors, reduced intensity conditioning regimens, and peripheral blood as source for stem cells (Arai et al., 2015, Biol Blood Marrow Transplant, 21:266-74). Due to an increased risk of non-relapse mortality, cGVHD remains the leading cause for late mortality following allogeneic HCT (Zeiser et al., 2018, Blood, 131:1399-405; Li et al., 2019, Br J Haematol, 184:323-36). Axatilimab is FDA approved for the treatment of cGVHD.

### SUMMARY

The present disclosure relates to antibodies that bind to colony stimulating factor 1 receptor (CSF-1R), compositions comprising those antibodies, and methods of using those antibodies.

In some embodiments, provided here is an antibody that binds to human colony stimulating factor 1 receptor (CSF-1R), the antibody comprising a variable heavy (VH) domain comprising VH complementarity determining region (CDR)1 (VHCDR1), VH CDR2, and VH CDR3, wherein: the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a variable light (VL) domain comprising VL CDR1, VL CDR2, and VL CDR3, wherein: the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7). In some embodiments, provided here is an antibody that binds to human colony stimulating factor 1 receptor (CSF-1R), the antibody comprising a variable heavy (VH) domain comprising VH complementarity determining region (CDR)1 (VHCDR1), VH CDR2, and VH CDR3, wherein: the VH CDR1 consists of the amino acid sequence GFSLTTYGMGVG (SEQ ID NO:2); the VH CDR2 consists of the amino acid sequence NIWWDDDKYYNPSLKN (SEQ ID NO:3); and the VH CDR3 consists of the amino acid sequence IGPIKYPTAPYRYFDF (SEQ ID NO:4); and wherein the antibody comprises a variable light (VL) domain comprising VL CDR1, VL CDR2, and VL CDR3, wherein: the VL CDR1 consists of the amino acid sequence LASEDIYDNLA (SEQ ID NO:5); the VL CDR2 consists of the amino acid sequence YASYLQD (SEQ ID NO: 6); and the VL CDR3 consists of the amino acid sequence LQDSEYPWT (SEQ ID NO: 7).

In some embodiments, the VH domain comprises the amino acid sequence EVTLKESGPALVKPTQTLTLTCTFSGFSLTTYGMGVGWIRQPPGKALEWLANIW WDDDKYYNPSLKNRLTISKDTSKNQVVLTMTNMDPVDTATYYCARIGPIKYPTA PYRYFDFWGQGTMVTVS (SEQ ID NO:8).

In some embodiments, the VL domain comprises the amino acid sequence DIQMTQSPSSLSASVGDRVTITCLASEDIYDNLAWYQQKPGKAPKLLIYYASYLQ DGVPSRFSGSGSGTDYTLTISSLQPEDFATYYCLQDSEYPWTFGGGTKVEIK (SEQ ID NO: 9).

In some embodiments, the VH domain comprises the amino acid sequence EVTLKESGPALVKPTQTLTLTCTFSGFSLTTYGMGVGWIRQPPGKALEWLANIW WDDDKYYNPSLKNRLTISKDTSKNQVVLTMTNMDPVDTATYYCARIGPIKYPTA PYRYFDFWGQGTMVTVS (SEQ ID NO:8), and wherein the VL domain comprises the amino acid sequence DIQMTQSPSSLSASVGDRVTITCLASEDIYDNLAWYQQKPGKAPKLLIYYASYLQ DGVPSRFSGSGSGTDYTLTISSLQPEDFATYYCLQDSEYPWTFGGGTKVEIK (SEQ ID NO: 9).

In some embodiments, the antibody comprises a heavy chain and a light chain, and wherein the heavy chain comprises the amino acid sequence set forth in SEQ ID NO: 10 and the light chain comprises the amino acid sequence set forth in SEQ ID NO:11.

In some embodiments, the antibody comprises a heavy chain and a light chain, and wherein the heavy chain comprises the amino acid sequence set forth in SEQ ID NO: 15 and the light chain comprises the amino acid sequence set forth in SEQ ID NO:11.

Also provided herein are nucleic acid sequence(s) encoding the heavy and/or light chain(s) of any one of the antibodies as described above. Also provided herein are expression vectors comprising the nucleic acid(s). Also provided herein are host cell comprising the expression vectors.

Also provided herein are pharmaceutical compositions comprising the antibody of any one of the antibodies as described above and at least one pharmaceutically acceptable diluent, carrier or excipient.

In some instances, provided herein are pharmaceutical compositions comprising a first antibody, a second antibody, and at least one pharmaceutically acceptable diluent, carrier or excipient, wherein the first antibody is any one of the antibodies described above, and wherein the second antibody is an antibody that binds to human colony stimulating factor 1 receptor (CSF-1R) comprises a variable heavy (VH) domain comprising VH complementarity determining region (CDR)1 (VHCDR1), VH CDR2, and VH CDR3, wherein: the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a variable light (VL) domain comprising VL CDR1, VL CDR2, and VL CDR3, wherein: the VL CDR1 comprises the amino acid sequence LASEDIYDNLA (SEQ ID NO:5); the VL CDR2 comprises the amino acid sequence YASSLQD (SEQ ID NO: 12); and the VL CDR3 comprises the amino acid sequence LQDSEYPWT (SEQ ID NO: 7). In some instances, provided herein are pharmaceutical compositions comprising a first antibody, a second antibody, and at least one pharmaceutically acceptable diluent, carrier or excipient, wherein the first antibody is any one of the antibodies described above, and wherein the second antibody is an antibody that binds to human colony stimulating factor 1 receptor (CSF-1R) comprises a variable heavy (VH) domain comprising VH complementarity determining region (CDR)1 (VHCDR1), VH CDR2, and VH CDR3, wherein: the VH CDR1 consists of the amino acid sequence GFSLTTYGMGVG (SEQ ID NO:2); the VH CDR2 consists of the amino acid sequence NIWWDDDKYYNPSLKN (SEQ ID NO:3); and the VH CDR3 consists of the amino acid sequence IGPIKYPTAPYRYFDF (SEQ ID NO:4); and wherein the antibody comprises a variable light (VL) domain comprising VL CDR1, VL CDR2, and VL CDR3, wherein: the VL CDR1 consists of the amino acid sequence LASEDIYDNLA (SEQ ID NO:5); the VL CDR2 consists of the amino acid sequence YASSLQD (SEQ ID NO: 12); and the VL CDR3 consists of the amino acid sequence LQDSEYPWT (SEQ ID NO: 7).

In some embodiments, the VH domain of the second antibody comprises the amino acid sequence EVTLKESGPALVKPTQTLTLTCTFSGFSLTTYGMGVGWIRQPPGKALEWLANIW WDDDKYYNPSLKNRLTISKDTSKNQVVLTMTNMDPVDTATYYCARIGPIKYPTA PYRYFDFWGQGTMVTVS (SEQ ID NO:8), and wherein the VL domain of the second antibody comprises DIQMTQSPSSLSASVGDRVTITCLASEDIYDNLAWYQQKPGKAPKLLIYYASSLQ DGVPSRFSGSGSGTDYTLTISSLQPEDFATYYCLQDSEYPWTFGGGTKVEIK (SEQ ID NO: 13).

In some embodiments, the second antibody comprises a heavy chain and a light chain, and wherein the heavy chain of the second antibody comprises the amino acid sequence set forth in SEQ ID NO:10 and the light chain the second antibody comprises the amino acid sequence set forth in SEQ ID NO: 14.

In some embodiments, the second antibody comprises a heavy chain and a light chain, and wherein the heavy chain of the second antibody comprises the amino acid sequence set forth in SEQ ID NO:15 and the light chain the second antibody comprises the amino acid sequence set forth in SEQ ID NO: 14.

In some embodiments, the VH domain of the first antibody comprises the amino acid sequence EVTLKESGPALVKPTQTLTLTCTFSGFSLTTYGMGVGWIRQPPGKALEWLANIW WDDDKYYNPSLKNRLTISKDTSKNQVVLTMTNMDPVDTATYYCARIGPIKYPTA PYRYFDFWGQGTMVTVS (SEQ ID NO:8), wherein the VL domain of the first antibody comprises DIQMTQSPSSLSASVGDRVTITCLASEDIYDNLAWYQQKPGKAPKLLIYYASYLQ DGVPSRFSGSGSGTDYTLTISSLQPEDFATYYCLQDSEYPWTFGGGTKVEIK (SEQ ID NO: 9), wherein the VH domain of the second antibody comprises the amino acid sequence EVTLKESGPALVKPTQTLTLTCTFSGFSLTTYGMGVGWIRQPPGKALEWLANIW WDDDKYYNPSLKNRLTISKDTSKNQVVLTMTNMDPVDTATYYCARIGPIKYPTA PYRYFDFWGQGTMVTVS (SEQ ID NO:8), and wherein the VL domain of the second antibody comprises DIQMTQSPSSLSASVGDRVTITCLASEDIYDNLAWYQQKPGKAPKLLIYYASSLQ DGVPSRFSGSGSGTDYTLTISSLQPEDFATYYCLQDSEYPWTFGGGTKVEIK (SEQ ID NO: 13).

In some embodiments, the first antibody comprises a heavy chain and a light chain, wherein the heavy chain of the first antibody comprises the amino acid sequence set forth in SEQ ID NO:10 and the light chain the first antibody comprises the amino acid sequence set forth in SEQ ID NO:11; and the second antibody comprises a heavy chain and a light chain, wherein the heavy chain of the second antibody comprises the amino acid sequence set forth in SEQ ID NO:10 and the light chain the second antibody comprises the amino acid sequence set forth in SEQ ID NO: 14.

In some embodiments, the first antibody comprises a heavy chain and a light chain, wherein the heavy chain of the first antibody comprises the amino acid sequence set forth in SEQ ID NO:15 and the light chain the first antibody comprises the amino acid sequence set forth in SEQ ID NO:11; and the second antibody comprises a heavy chain and a light chain, wherein the heavy chain of the second antibody comprises the amino acid sequence set forth in SEQ ID NO:15 and the light chain the second antibody comprises the amino acid sequence set forth in SEQ ID NO:14.

In some embodiments, the first antibody is present in an amount less than about 5.0%, less than about 4.5%, less than about 4.0%, less than about 3.5%, less than about 3.0%, less than about 2.5%, less than about 2.0%, less than about 1.5%, less than about 1.0%, or less than about 0.5% of the total amount of antibody in the pharmaceutical composition.

In some embodiments, the first antibody is present in an amount of 0.5% to 5.0% of the total amount of antibody in the pharmaceutical composition.

In some embodiments, the first antibody is present in an amount of 2.0% to 3.0% of the total amount of antibody in the pharmaceutical composition.

In some embodiments, the first antibody is present in an amount of about 2.5% of the total amount of antibody in the pharmaceutical composition.

In some embodiments, the first antibody is present in an amount of 0.5% to 5.0% of the total amount of antibody in the pharmaceutical composition, and wherein the second antibody is present in an amount of 95.0% to 99.5% of the total amount of antibody in the pharmaceutical composition.

In some embodiments, the first antibody is present in an amount of 2.0% to 3.0% of the total amount of antibody in the pharmaceutical composition, and wherein the second antibody is present in an amount of 97.0% to 98.0% of the total amount of antibody in the pharmaceutical composition.

In some embodiments, the first antibody is present in an amount of about 2.5% of the total amount of antibody in the pharmaceutical composition, and wherein the second antibody is present in an amount of about 97.5% of the total amount of antibody in the pharmaceutical composition.

Also provided herein are methods of treating chronic graft-versus-host disease (cGVHD) in a human subject in need thereof, the method comprising administering to the human subject a therapeutically effective amount of any one of the antibodies as described above or any one of the pharmaceutical compositions as described above.

In some embodiments, the antibody or pharmaceutical composition is administered intravenously once every two weeks at a total antibody dose of 0.3 mg per kg of body weight of the human subject.

In some embodiments, (i) the human subject is an adult patient or a pediatric patient with a body weight greater than about 55 kg and the antibody or pharmaceutical composition is administered intravenously once every two weeks at a total antibody dose of about 22 mg; (ii) the human subject is a pediatric patient with a body weight greater than or equal to about 30 kg and less than or equal to about 55 kg and the antibody or pharmaceutical composition is administered intravenously once every two weeks at a total antibody dose of about 14 mg; or (iii) the human subject is a pediatric patient with a body weight less than about 30 kg and the antibody or pharmaceutical composition is administered intravenously once every two weeks at a total antibody dose of about 9 mg.

In some embodiments, (i) the human subject has a body weight greater than about 60 kg and the antibody or pharmaceutical composition is administered intravenously once every two weeks at a total antibody dose of about 23 mg; (ii) the human subject has a body weight greater than or equal to about 40 kg and less than or equal to about 60 kg and the antibody or pharmaceutical composition is administered intravenously once every two weeks at a total antibody dose of about 17 mg; or (iii) the human subject has a body weight less than about 40 kg and the antibody or pharmaceutical composition is administered intravenously once every two weeks at a total antibody dose of about 10.5 mg. In some instances, the antibody or pharmaceutical composition is administered after failure of at least two prior lines of systemic therapy.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a graph showing antibody potency measured by CSF-1R binding ELISA.

FIG. 2 is a graph showing antibody potency measured by a CSF-1R blockade bioassay.

FIG. 3 is a graph showing the impact of the amount of the S53Y sequence variant on antibody potency measured by CSF-1R binding ELISA with linear regression.

### DETAILED DESCRIPTION

The present disclosure provides antibodies that bind to human colony stimulating factor 1 receptor (CSF-1R) and methods of treating cGVHD in a human subject in need thereof by administering the antibodies to the human subject.

CSF-1R is a receptor for the cytokine colony stimulating factor 1 (CSF-1), which is responsible for the production, differentiation, and function of macrophages.

The amino acid sequence of the human CSF-1R protein is:

(SEQ ID NO: 1)
MGPGVLLLLLVATAWHGQGIPVIEPSVPELVVKPGATVTLRCVGN
GSVEWDGPPSPHWTLYSDGSSSILSTNNATFQNTGTYRCTEPGDP
LGGSAAIHLYVKDPARPWNVLAQEVVVFEDQDALLPCLLTDPVLE
AGVSLVRVRGRPLMRHTNYSFSPWHGFTIHRAKFIQSQDYQCSAL
MGGRKVMSISIRLKVQKVIPGPPALTLVPAELVRIRGEAAQIVCS
ASSVDVNFDVFLQHNNTKLAIPQQSDFHNNRYQKVLTLNLDQVDF
QHAGNYSCVASNVQGKHSTSMFFRVVESAYLNLSSEQNLIQEVTV
GEGLNLKVMVEAYPGLQGENWTYLGPFSDHQPEPKLANATTKDTY
RHTFTLSLPRLKPSEAGRYSFLARNPGGWRALTFELTLRYPPEVS
VIWTFINGSGTLLCAASGYPQPNVTWLQCSGHTDRCDEAQVLQVW
DDPYPEVLSQEPFHKVTVQSLLTVETLEHNQTYECRAHNSVGSGS
WAFIPISAGAHTHPPDEFLFTPVVVACMSIMALLLLLLLLLLYKY
KQKPKYQVRWKIIESYEGNSYTFIDPTQLPYNEKWEFPRNNLQFG
KTLGAGAFGKVVEATAFGLGKEDAVLKVAVKMLKSTAHADEKEAL
MSELKIMSHLGQHENIVNLLGACTHGGPVLVITEYCCYGDLLNFL
RRKAEAMLGPSLSPGQDPEGGVDYKNIHLEKKYVRRDSGFSSQGV
DTYVEMRPVSTSSNDSFSEQDLDKEDGRPLELRDLLHESSQVAQG
MAFLASKNCIHRDVAARNVLLTNGHVAKIGDFGLARDIMNDSNYI
VKGNARLPVKWMAPESIFDCVYTVQSDVWSYGILLWEIFSLGLNP
YPGILVNSKFYKLVKDGYQMAQPAFAPKNIYSIMQACWALEPTHR
PTFQQICSFLQEQAQEDRRERDYTNLPSSSRSGGSGSSSSELEEE
SSSEHLTCCEQGDIAQPLLQPNNYQFC.

Anti-CSF-1R antibodies can be used to treat cGVHD as described herein.

### Anti-Colony Stimulating Factor 1 Receptor Antibodies

The present disclosure provides a pharmaceutical composition comprising an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7).

In some embodiments, a pharmaceutical composition described herein comprises an anti-CSF-1R antibody comprising a VH domain comprising the amino acid sequence set forth in SEQ ID NO:8 and a VL domain comprising the amino acid sequence set forth in SEQ ID NO:13.

In some embodiments, a pharmaceutical composition described herein comprises an anti-CSF-1R antibody comprising a heavy chain comprising the amino acid sequence set forth in SEQ ID NO:10 and a light chain comprising the amino acid sequence set forth in SEQ ID NO:14.

In some embodiments, a pharmaceutical composition described herein comprises axatilimab.

Axatilimab (also known as SNDX-6352) is a humanized IgG4 monoclonal antibody that binds to human CSF-1R and inhibits its function. Axatilimab is described in U.S. Pat. No. 9,908,939, which is incorporated by reference in its entirety.

The amino acid sequences of axatilimab heavy and light chains are shown below. Complementarity-determining regions (CDRs) 1, 2, and 3 of the variable heavy (VH) domain and the variable light (VL) domain are shown in that order from N-terminus to the C-terminus of the mature VL and VH sequences and are both underlined and boldened. With the exception of VH CDR-1, the CDRs were defined according to Kabat. The VH CDR-1 is defined according to the combined Kabat-Chothia definitions. Variable regions are underlined. An antibody comprising of the heavy chain (SEQ ID NO: 10) and the light chain (SEQ ID NO:14) listed below is termed axatilimab.

Axatilimab Heavy Chain:
(SEQ ID NO: 10)
EVTLKESGPALVKPTQTLTLTCTFSGFSLTTYGMGVGWIRQPPGK
ALEWLANIWWDDDKYYNPSLKNRLTISKDTSKNQVVLTMTNMDPV
DTATYYCARIGPIKYPTAPYRYFDFWGQGTMVTVSSASTKGPSVF
PLAPCSRSTSESTAALGCLVKDYFPEPVTVSWNSGALTSGVHTFP
AVLQSSGLYSLSSVVTVPSSSLGTKTYTCNVDHKPSNTKVDKRVE
SKYGPPCPPCPAPEFLGGPSVFLFPPKPKDTLMISRTPEVTCVVV
DVSQEDPEVQFNWYVDGVEVHNAKTKPREEQFNSTYRVVSVLTVL
HQDWLNGKEYKCKVSNKGLPSSIEKTISKAKGQPREPQVYTLPPS
QEEMTKNQVSLTCLVKGFYPSDIAVEWESNGQPENNYKTTPPVLD
SDGSFFLYSRLTVDKSRWQEGNVFSCSVMHEALHNHYTQKSLSLS
LGK.
Axatilimab Light Chain:
(SEQ ID NO: 14)
DIQMTQSPSSLSASVGDRVTITCLASEDIYDNLAWYQQKPGKAPK
LLIYYASSLQDGVPSRESGSGSGTDYTLTISSLQPEDFATYYCLQ
DSEYPWTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC

The variable heavy (VH) domain of axatilimab has the following amino acid sequence:

(SEQ ID NO: 8)
EVTLKESGPALVKPTQTLTLTCTFSGFSLTTYGMGVGWIRQPPGK
ALEWLANIWWDDDKYYNPSLKNRLTISKDTSKNQVVLTMTNMDPV
DTATYYCARIGPIKYPTAPYRYFDFWGQGTMVTVS

The variable light (VL) domain of axatilimab has the following amino acid sequence:

(SEQ ID NO: 13)
DIQMTQSPSSLSASVGDRVTITCLASEDIYDNLAWYQQKPGKAPK
LLIYYASSLQDGVPSRESGSGSGTDYTLTISSLQPEDFATYYCLQ
DSEYPWTFGGGTKVEIK.

The amino acid sequences of the VH CDRs of axatilimab are listed below:

VH CDR1:
(SEQ ID NO: 2)
GFSLTTYGMGVG;
VH CDR2:
(SEQ ID NO: 3)
NIWWDDDKYYNPSLKN;
and
VH CDR3:
(SEQ ID NO : 4)
IGPIKYPTAPYRYFDF.

The amino acid sequences of the VL CDRs of axatilimab are listed below:

VL CDR1:
(SEQ ID NO: 5)
LASEDIYDNLA;
VL CDR2:
(SEQ ID NO: 12)
YASSLQD;
and
VL CDR3:
(SEQ ID NO: 7)
LQDSEYPWT.

During the manufacturing of axatilimab, a sequence variant was identified. The amino acid sequences of the heavy and light chains of the sequence variant of axatilimab are shown below. Complementarity-determining regions (CDRs) 1, 2, and 3 of the variable heavy (VH) domain and the variable light (VL) domain are shown in that order from N-terminus to the C-terminus of the mature VL and VH sequences and are both underlined and boldened. With the exception of VH CDR-1, the CDRs were defined according to Kabat. The VH CDR-1 is defined according to the combined Kabat-Chothia definitions. Variable regions are underlined.

Axatilimab-Sequence Variant Heavy Chain:
(SEQ ID NO: 10)
EVTLKESGPALVKPTQTLTLTCTFSGFSLTTYGMGVGWIRQPPGK
ALEWLANIWWDDDKYYNPSLKNRLTISKDTSKNQVVLTMTNMDPV
DTATYYCARIGPIKYPTAPYRYFDFWGQGTMVTVSSASTKGPSVF
PLAPCSRSTSESTAALGCLVKDYFPEPVTVSWNSGALTSGVHTFP
AVLQSSGLYSLSSVVTVPSSSLGTKTYTCNVDHKPSNTKVDKRVE
SKYGPPCPPCPAPEFLGGPSVFLFPPKPKDTLMISRTPEVTCVVV
DVSQEDPEVQFNWYVDGVEVHNAKTKPREEQFNSTYRVVSVLTVL
HQDWLNGKEYKCKVSNKGLPSSIEKTISKAKGQPREPQVYTLPPS
QEEMTKNQVSLTCLVKGFYPSDIAVEWESNGQPENNYKTTPPVLD
SDGSFFLYSRLTVDKSRWQEGNVFSCSVMHEALHNHYTQKSLSLS
LGK.
Axatilimab-Sequence Variant Light Chain:
(SEQ ID NO: 11)
DIQMTQSPSSLSASVGDRVTITCLASEDIYDNLAWYQQKPGKAPK
LLIYYASYLQDGVPSRESGSGSGTDYTLTISSLQPEDFATYYCLQ
DSEYPWTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC.

The variable heavy (VH) domain of axatilimab-sequence variant has the following amino acid sequence:

(SEQ ID NO: 8)
EVTLKESGPALVKPTQTLTLTCTFSGFSLTTYGMGVGWIRQPPGK
ALEWLANIWWDDDKYYNPSLKNRLTISKDTSKNQVVLTMTNMDPV
DTATYYCARIGPIKYPTAPYRYFDFWGQGTMVTVS

The variable light (VL) domain of axatilimab-sequence variant has the following amino acid sequence:

(SEQ ID NO: 9)
DIQMTQSPSSLSASVGDRVTITCLASEDIYDNLAWYQQKPGKAPK
LLIYYASYLQDGVPSRESGSGSGTDYTLTISSLQPEDFATYYCLQ
DSEYPWTFGGGTKVEIK.

The amino acid sequences of the VH CDRs of axatilimab-sequence variant are listed below:

VH CDR1:
(SEQ ID NO: 2)
GFSLTTYGMGVG;
VH CDR2:
(SEQ ID NO: 3)
NIWWDDDKYYNPSLKN;
and
VH CDR3:
(SEQ ID NO: 4)
IGPIKYPTAPYRYFDF.

The amino acid sequences of the VL CDRs of axatilimab are listed below:

VL CDR1:
(SEQ ID NO: 5)
LASEDIYDNLA;
VL CDR2:
(SEQ ID NO: 6)
YASYLQD;
and
VL CDR3:
(SEQ ID NO: 7)
LQDSEYPWT.

The present disclosure provides an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7).

In some embodiments, an anti-CSF-1R antibody described herein comprises a VH domain comprising the amino acid sequence set forth in SEQ ID NO:8 and a VL domain comprising the amino acid sequence set forth in SEQ ID NO:9.

In some embodiments, an anti-CSF-1R antibody described herein comprises a heavy chain comprising the amino acid sequence set forth in SEQ ID NO:10 and a light chain comprising the amino acid sequence set forth in SEQ ID NO:11.

In some embodiments, an anti-CSF-1R antibody described herein comprises a first variable region and a second variable region, wherein the first variable region comprises a first VH domain and a first VL domain, wherein the second variable region comprises a second VH domain and a second VL domain, wherein the first VH domain comprises VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4), wherein the first VL domain comprises VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7), wherein the second VH domain comprises VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4), wherein the second VL domain comprises VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7).

In some embodiments, an anti-CSF-1R antibody described herein comprises a first variable region and a second variable region, wherein the first variable region comprises a first VH domain and a first VL domain, wherein the second variable region comprises a second VH domain and a second VL domain, wherein the first VH domain comprises VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4), wherein the first VL domain comprises VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7), wherein the second VH domain comprises VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4), wherein the second VL domain comprises VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7).

In some embodiments, an anti-CSF-1R antibody described herein comprises a first variable region and a second variable region, wherein the first variable region comprises a first VH domain and a first VL domain, wherein the second variable region comprises a second VH domain and a second VL domain, wherein the first VH domain comprises VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4), wherein the first VL domain comprises VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7), wherein the second VH domain comprises VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4), wherein the second VL domain comprises VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7).

In some embodiments, an anti-CSF-1R antibody described herein comprises a first variable region and a second variable region, wherein the first variable region comprises a first VH domain and a first VL domain, wherein the second variable region comprises a second VH domain and a second VL domain, wherein the first VH domain comprises the amino acid sequence set forth in SEQ ID NO:8, wherein the first VL domain comprises the amino acid sequence set forth in SEQ ID NO:9, wherein the second VH domain comprises the amino acid sequence set forth in SEQ ID NO:8, and wherein the second VL domain comprises the amino acid sequence set forth in SEQ ID NO: 9.

In some embodiments, an anti-CSF-1R antibody described herein comprises a first variable region and a second variable region, wherein the first variable region comprises a first VH domain and a first VL domain, wherein the second variable region comprises a second VH domain and a second VL domain, wherein the first VH domain comprises the amino acid sequence set forth in SEQ ID NO:8, wherein the first VL domain comprises the amino acid sequence set forth in SEQ ID NO:9, wherein the second VH domain comprises the amino acid sequence set forth in SEQ ID NO:8, and wherein the second VL domain comprises the amino acid sequence set forth in SEQ ID NO: 13.

In some embodiments, an anti-CSF-1R antibody described herein comprises a first variable region and a second variable region, wherein the first variable region comprises a first VH domain and a first VL domain, wherein the second variable region comprises a second VH domain and a second VL domain, wherein the first VH domain comprises the amino acid sequence set forth in SEQ ID NO:8, wherein the first VL domain comprises the amino acid sequence set forth in SEQ ID NO:13, wherein the second VH domain comprises the amino acid sequence set forth in SEQ ID NO:8, and wherein the second VL domain comprises the amino acid sequence set forth in SEQ ID NO: 13.

In some embodiments, an anti-CSF-1R antibody described herein comprises a first heavy chain, a first light chain, a second heavy chain, and a second light chain, wherein the first heavy chain comprises the amino acid sequence set forth in SEQ ID NO: 10, wherein the first light chain comprises the amino acid sequence set forth in SEQ ID NO: 11, wherein the second heavy chain comprises the amino acid sequence set forth in SEQ ID NO:10, and wherein the second light chain comprises the amino acid sequence set forth in SEQ ID NO:11.

In some embodiments, an anti-CSF-1R antibody described herein comprises a first heavy chain, a first light chain, a second heavy chain, and a second light chain, wherein the first heavy chain comprises the amino acid sequence set forth in SEQ ID NO: 10, wherein the first light chain comprises the amino acid sequence set forth in SEQ ID NO: 11, wherein the second heavy chain comprises the amino acid sequence set forth in SEQ ID NO:10, and wherein the second light chain comprises the amino acid sequence set forth in SEQ ID NO:14.

In some embodiments, an anti-CSF-1R antibody described herein comprises a first heavy chain, a first light chain, a second heavy chain, and a second light chain, wherein the first heavy chain comprises the amino acid sequence set forth in SEQ ID NO: 10, wherein the first light chain comprises the amino acid sequence set forth in SEQ ID NO: 14, wherein the second heavy chain comprises the amino acid sequence set forth in SEQ ID NO:10, and wherein the second light chain comprises the amino acid sequence set forth in SEQ ID NO:14.

In certain embodiments, the binding affinity (K_{D}), on-rate (K_{D} on) and/or off-rate (K_{D} off) of the an anti-CSF-1R antibody described herein is similar to that of axatilimab, e.g., having a difference of less than about 5-fold, 2-fold, 1-fold (100%), 50%, 30%, 20%, 10%, 5%, 3%, 2% or 1%.

In certain embodiments, the potency of an anti-CSF-1R antibody described herein, as measured by a CSF-1R competitive binding ELISA is similar to that of axatilimab, e.g., having a difference of less than about 5-fold, 2-fold, 1-fold (100%), 50%, 30%, 20%, 10%, 5%, 3%, 2% or 1%. See Examples 1 and 2 below.

In certain embodiments, the binding affinity (K_{D}), on-rate (K_{D} on) and/or off-rate (K_{D} off) for FcRn, FcγRI, FcγRIIa (R and H variants), FcγRIIIa (F and V variants) and C1q of an anti-CSF-1R antibody described herein is similar to that of axatilimab, e.g., having a difference of less than about 5-fold, 2-fold, 1-fold (100%), 50%, 30%, 20%, 10%, 5%, 3%, 2% or 1%. See Example 3 below.

In certain embodiments, the safety and immunogenicity, as measured by the number of treatment emergent adverse event or the number of adverse drug reactions, of an anti-CSF-1R antibody described herein is similar to that of axatilimab, e.g., having a difference of less than about 5-fold, 2-fold, 1-fold (100%), 50%, 30%, 20%, 10%, 5%, 3%, 2% or 1%. See Examples 3 and 4 below.

In certain embodiments, an anti-CSF-1R antibody described herein includes a human heavy chain and light chain constant region. In certain embodiments, the heavy chain constant region comprises a CH1 domain and a hinge region. In some embodiments, the heavy chain constant region comprises a CH2 domain. In some embodiments, the heavy chain constant region comprises a CH3 domain. In some embodiments, the heavy chain constant region comprises CH1, CH2 and CH3 domains. If the heavy chain constant region includes substitutions, such substitutions can modify the properties of the antibody (e.g., increase or decrease one or more of: Fc receptor binding, antibody glycosylation, the number of cysteine residues, effector cell function, or complement function). In certain embodiments, the antibody is an IgG antibody. In specific embodiments, the antibody is selected from the group consisting of IgG1, IgG2, IgG3, and IgG4.

In some embodiments, an anti-CSF-1R antibody is provided wherein a C-terminal residue of an antibody sequence described herein is cleaved, for example, the C-terminal residue of a heavy chain sequence, for example, a terminal lysine. Generally, the cleavage results from post-translation modifications of the expressed antibody. For example, an anti-CSF-1R antibody can include: (i) a heavy chain comprising the amino acid sequence set forth in SEQ ID NO: 15 (below) and a light chain comprising the amino acid sequence set forth in SEQ ID NO:14; or (ii) a heavy chain comprising the amino acid sequence set forth in SEQ ID NO:15 (below) and a light chain comprising the amino acid sequence set forth in SEQ ID NO:11.

(SEQ ID NO: 15)
EVTLKESGPALVKPTQTLTLTCTFSGFSLTTYGMGVGWIRQPPGKA
LEWLANIWWDDDKYYNPSLKNRLTISKDTSKNQVVLTMTNMDPVD
TATYYCARIGPIKYPTAPYRYFDFWGQGTMVTVSSASTKGPSVFP
LAPCSRSTSESTAALGCLVKDYFPEPVTVSWNSGALTSGVHTFPA
VLQSSGLYSLSSVVTVPSSSLGTKTYTCNVDHKPSNTKVDKRVES
KYGPPCPPCPAPEFLGGPSVFLFPPKPKDTLMISRTPEVTCVVVD
VSQEDPEVQFNWYVDGVEVHNAKTKPREEQFNSTYRVVSVLTVLH
QDWLNGKEYKCKVSNKGLPSSIEKTISKAKGQPREPQVYTLPPSQ
EEMTKNQVSLTCLVKGFYPSDIAVEWESNGQPENNYKTTPPVLDS
DGSFFLYSRLTVDKSRWQEGNVFSCSVMHEALHNHYTQKSLSLSL
G.

### Bispecific Antibodies

In certain embodiments, an anti-CSF-1R antibody described herein (e.g., axatilimab, an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7), or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) is present in a bispecific antibody.

Bispecific antibodies are antibodies that have binding specificities for at least two different epitopes. Exemplary bispecific antibodies may bind to two different epitopes of the CSF-1R protein. Other such antibodies may combine a CSF-1R binding site with a binding site for another protein. Bispecific antibodies can be prepared as full length antibodies or low molecular weight forms thereof (e.g., F(ab′)₂ bispecific antibodies, sc(Fv)2 bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

### Multivalent Antibodies

In certain embodiments, an anti-CSF-1R antibody thereof described herein (e.g., axatilimab, an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7), or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) is present in a multivalent antibody.

A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Conjugated Antibodies

The anti-CSF-1R antibodies disclosed herein (e.g., axatilimab, an anti-CSF-1R antibody comprising a VH domain comprising VH CDR1, VH CDR2, and VH CDR3, wherein the VH CDR1 comprises the amino acid sequence GFSLTTYGMGVG (SEQ ID NO: 2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7), or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO: 2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) may be conjugated antibodies which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g., 90Y, 1311) fluorescent substances, luminescent substances, haptens, enzymes, metal chelates, drugs, and toxins (e.g., calicheamicin, Pseudomonas exotoxin A, ricin (e.g., deglycosylated ricin A chain), exatecan, auristatins (e.g., auristatin E), maytansine, pyrrolobenzodiazepine (PBD)).

In one embodiment, to improve the cytotoxic actions of anti-CSF-1R antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-CSF-1R antibody is modified with a moiety that improves its stabilization and/or retention in circulation, e.g., in blood, serum, or other tissues, e.g., by at least about 1.5, 2, 5, 10, or 50 fold. For example, the anti-CSF-1R antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-CSF-1R antibody can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies or the lower molecular weight forms thereof described herein. Methods for modifying antibodies are well known in the art (e.g., U.S. Pat. Nos. 5,057,313 and 5,156,840).

### Methods of Making and Producing Antibodies

Antibodies can be made, for example, by preparing and expressing synthetic genes that encode the recited amino acid sequences or by mutating human germline genes to provide a gene that encodes the recited amino acid sequences. Moreover, this antibody and other anti-CSF-1R antibodies can be obtained, e.g., using one or more of the following methods.

Humanized antibodies can be generated by replacing sequences of the Fv variable region that are not directly involved in antigen binding with equivalent sequences from human Fv variable regions. General methods for generating humanized antibodies are provided by Morrison, S. L., Science, 229:1202-1207 (1985), by Oi et al., BioTechniques, 4:214 (1986), and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al., J. Mol. Biol., 227:776-798 (1992); Cook, G. P. et al., Immunol. Today, 16:237-242 (1995); Chothia, D. et al., J. Mol. Bio. 227:799-817 (1992); and Tomlinson et al., EMBO J., 14:4628-4638 (1995). The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and U.S. Pat. No. 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

The anti-CSF-1R antibodies can be in the form of full length antibodies, or in the form of low molecular weight forms (e.g., biologically active antibody fragments or minibodies) of the anti-CSF-1R antibodies, e.g., Fab, Fab′, F(ab′)₂, Fv, Fd, dAb, scFv, and sc(Fv)2. Other anti-CSF-1R antibodies encompassed by this disclosure include single domain antibody (sdAb) containing a single variable chain such as, VH or VL, or a biologically active fragment thereof. See, e.g., Moller et al., J. Biol. Chem., 285 (49): 38348-38361 (2010); Harmsen et al., Appl. Microbiol. Biotechnol., 77 (1): 13-22 (2007); U.S. 2005/0079574 and Davies et al. (1996) Protein Eng., 9 (6): 531-7. Like a whole antibody, a sdAb is able to bind selectively to a specific antigen. With a molecular weight of only 12-15 kDa, sdAbs are much smaller than common antibodies and even smaller than Fab fragments and single-chain variable fragments.

Antibodies may be produced in, for example, bacterial or eukaryotic cells. Some antibodies, e.g., Fab's, can be produced in bacterial cells, e.g., E. coli cells. Antibodies can also be produced in eukaryotic cells such as transformed cell lines (e.g., CHO, 293E, COS). In addition, antibodies (e.g., scFv's) can be expressed in a yeast cell such as Pichia (see, e.g., Powers et al., J Immunol Methods. 251:123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), human embryonic kidney 293 cells (e.g., 293, 293E, 293T), COS cells, NIH3T3 cells, lymphocytic cell lines, e.g., NSO myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-CSF-1R antibody is introduced into dhfr CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Antibody Pharmaceutical Compositions and Administration

An anti-CSF-1R antibody described herein (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO: 3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) can be formulated as a pharmaceutical composition for administration to a human subject, e.g., to treat a disorder described herein.

Also provided herein are pharmaceutical compositions comprising a mixture of at least two different species of anti-CSF-1R antibodies. In some embodiments provided herein are pharmaceutical compositions comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)) and a sequence variant of axatilimab (e.g., a sequence variant of axatilimab containing the S53Y substitution as described above).

A mixture of at least two different species of anti-CSF-1R antibodies (e.g., a first anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7), and a second anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)) can be formulated as a pharmaceutical composition for administration to a human subject, e.g., to treat a disorder described herein.

In certain embodiments, the first antibody is present in an amount less than about 5.0%, less than about 4.5%, less than about 4.0%, less than about 3.5%, less than about 3.0%, less than about 2.5%, less than about 2.0%, less than about 1.5%, less than about 1.0%, or less than about 0.5% of the total amount of antibody in the pharmaceutical composition.

In certain embodiments, the first antibody is present in an amount of 0.5% to 5.0% of the total amount of antibody in the pharmaceutical composition.

In certain embodiments, the first antibody is present in an amount of 2.0% to 3.0% of the total amount of antibody in the pharmaceutical composition.

In certain embodiments, the first antibody is present in an amount of about 2.5% of the total amount of antibody in the pharmaceutical composition.

In certain embodiments, the first antibody is present in an amount of 0.5% to 5.0% of the total amount of antibody in the pharmaceutical composition, and the second antibody is present in an amount of 95.0% to 99.5% of the total amount of antibody in the pharmaceutical composition.

In certain embodiments, the first antibody is present in an amount of 2.0% to 3.0% of the total amount of antibody in the pharmaceutical composition, and the second antibody is present in an amount of 97.0% to 98.0% of the total amount of antibody in the pharmaceutical composition.

In certain embodiments, the first antibody is present in an amount of about 2.5% of the total amount of antibody in the pharmaceutical composition, and the second antibody is present in an amount of about 97.5% of the total amount of antibody in the pharmaceutical composition.

Typically, a pharmaceutical composition includes a pharmaceutically acceptable diluent, carrier or excipient. As used herein, pharmaceutically acceptable carrier includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X). Suitable anti-CSF-1R antibody formulations are described, e.g., in U.S. Pat. No. 10,039,826 B2, which is incorporated by reference in its entirety.

The anti-CSF-1R antibodies and pharmaceutical compositions as described herein (e.g., a pharmaceutical composition comprising a mixture of axatilimab and an axatilimab-sequence variant) can be administered to a human subject having cGVHD. In some embodiments, the anti-CSF-1R antibodies and pharmaceutical compositions as described herein (e.g., a pharmaceutical composition comprising a mixture of axatilimab and an axatilimab-sequence variant) can be administered to a human subject having recurrent or refractory active cGVHD. In some embodiments, the anti-CSF-1R antibodies and pharmaceutical compositions as described herein (e.g., a pharmaceutical composition comprising a mixture of axatilimab and an axatilimab-sequence variant) can be administered to a human subject having recurrent or refractory active cGVHD after two or more lines of systemic therapy (e.g., ruxolitinib, belumosudil, or ibrutinib).

The anti-CSF-1R antibodies and pharmaceutical compositions as described herein (e.g., a pharmaceutical composition comprising a mixture of axatilimab and an axatilimab-sequence variant) can be administered to a human subject having cGVHD who has received one or more previous cGVHD treatments. In some embodiments, the human subject has received at least two previous cGVHD treatments. In some embodiments, the human subject has received at least three previous cGVHD treatments. In some embodiments, the human subject has received at least four previous cGVHD treatments. In some embodiments, the human subject has received at least five previous cGVHD treatments. In some embodiments, the human subject has received at least six previous cGVHD treatments.

The anti-CSF-1R antibodies and pharmaceutical compositions as described herein (e.g., a pharmaceutical composition comprising a mixture of axatilimab and an axatilimab-sequence variant) can be administered to a human subject after failure of at least two prior lines of systemic therapy, which can include one or more of ruxolitinib, belumosudil, and ibrutinib. In some embodiments, the human subject was prior treated with ibrutinib. In some embodiments, the human subject was prior treated with ruxolitinib. In some embodiments, the human subject was prior treated with belumosudil.

The anti-CSF-1R antibodies and pharmaceutical compositions as described herein (e.g., a pharmaceutical composition comprising a mixture of axatilimab and an axatilimab-sequence variant) can be administered to a human subject by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the anti-CSF-1R antibodies and pharmaceutical compositions as described herein (e.g., a pharmaceutical composition comprising a mixture of axatilimab and an axatilimab-sequence variant) can also be tailored for a human subject, e.g., by monitoring the human subject, e.g., using tomographic imaging, e.g., to visualize an organ.

The anti-CSF-1R antibodies and pharmaceutical compositions as described herein (e.g., a pharmaceutical composition comprising a mixture of axatilimab and an axatilimab-sequence variant) can be administered in a mg/kg subject weight dose (as used herein, “mg/kg” refers to total amount of antibody (e.g., a combination of axatilimab and an axatilimab-sequence variant) in milligrams administered per kilogram of body weight of the treated subject).

A human subject to be treated according to methods described herein can be an adult patient or a pediatric patient. As used herein, “an adult patient” refers to a human subject that is 18 years of age or older. As used herein, “a pediatric patient” or “a pediatric human subject” refers to a human subject that is under 18 years of age. In some embodiments, a pediatric patient or a pediatric human subject can include a human subject who is 6 years of age or older. In some embodiments, a pediatric patient or a pediatric human subject can include a human subject who is aged from birth through the first 28 days of life; from 29 days to less than 2 years old; from 2 years to less than 12 years old; or from 12 years to 17 years old.

An exemplary weight-based dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO: 2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) to a human subject (e.g., an adult patient), at a dosage of about 0.3 mg/kg once every two weeks.

As used herein, “about” when referring to a measurable value such as an amount, a dosage, a temporal duration, a percentage, and the like, is meant to encompass variations of ±10%. For example, with respect to doses or dosages, the term “about” denotes a range that is ±10% of a recited dose, such that, for example, a dose of about 0.3 mg/kg will be between 0.27 mg/kg and 0.33 mg/kg subject weight. Exemplary doses included within “about 0.3 mg/kg” are 0.27 mg/kg, 0.28 mg/kg, 0.29 mg/kg, 0.3 mg/kg, 0.31 mg/kg, 0.32 mg/kg, and 0.33 mg/kg. Exemplary doses included within “about 0.35 mg/kg” are 0.32 mg/kg, 0.33 mg/kg, 0.34 mg/kg, 0.35 mg/kg, 0.36 mg/kg, 0.37 mg/kg, and 0.38 mg/kg. In another example, a body weight of “about 55 kg” encompasses 49.5 kg to 60.5 kg and a dose of “about 22 mg” encompasses 19.8 mg to 24.2 mg. Exemplary body weights included within “about 55 kg” are 50 kg, 51 kg, 52 kg, 53 kg, 54 kg, 55 kg, 56 kg, 57 kg, 58 kg, 59 kg, and 60 kg. Exemplary doses included within “about 22 mg” are 20 mg, 21 mg, 22 mg, 23 mg, and 24 mg. In another example, a percentage of “about 2.5%” encompasses 2.25% to 2.75%.

A further exemplary weight-based dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)) to a human subject (e.g., an adult patient) at a dosage of 0.3 mg/kg once every two weeks.

An exemplary weight-based dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO: 2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) to a human subject (e.g., pediatric patient) at a dosage of about 0.35 mg/kg once every two weeks.

A further exemplary weight-based dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)) to a human subject (e.g., pediatric patient) at a dosage of 0.35 mg/kg once every two weeks.

The anti-CSF-1R antibodies, as described herein (e.g., a mixture of axatilimab and an axatilimab-sequence variant) can be administered in a flat dose (as used herein, “flat dose” refers to total amount of antibody (e.g., a combination of axatilimab and an axatilimab-sequence variant) in milligrams administered to all treated subjects regardless of their weight, age or other factors).

An exemplary flat dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO: 2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) to a human subject at a dosage of about 22 mg once every two weeks.

A further exemplary flat dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO: 2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) to a human subject at a dosage of 22 mg once every two weeks.

The anti-CSF-1R antibodies, as described herein (e.g., a mixture of axatilimab and an axatilimab-sequence variant) can be administered in a weight-band dose. As used herein, “weight-band dose” refers to total amount of antibody (e.g., a combination of axatilimab and an axatilimab-sequence variant) in milligrams administered per pre-defined ranges (or bands) of body weight of the treated subject.

An exemplary weight-band dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO: 2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) at a dosage of (i) about 22 mg once every two weeks to an adult patient or a pediatric patient with a body weight greater than about 55 kg; (ii) about 14 mg once every two weeks to a pediatric patient with a body weight greater than or equal to about 30 kg and less than or equal to about 55 kg; or (iii) about 9 mg once every two weeks to a pediatric patient with a body weight less than about 30 kg. In some embodiments, the pediatric patient is 6 years of age or older.

A further exemplary weight-band dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)), after failure of at least two prior lines of systemic therapy, at a dosage of (i) about 22 mg once every two weeks to an adult patient or a pediatric patient with a body weight greater than about 55 kg; (ii) about 14 mg once every two weeks to a pediatric patient with a body weight greater than or equal to about 30 kg and less than or equal to about 55 kg; or (iii) about 9 mg once every two weeks to a pediatric patient with a body weight less than about 30 kg. In some embodiments, the pediatric patient is 6 years of age or older.

A further exemplary weight-band dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)) at a dosage of (i) 22 mg once every two weeks to an adult patient or a pediatric patient with a body weight greater than 55 kg; (ii) 14 mg once every two weeks to a pediatric patient with a body weight greater than or equal to about 30 kg and less than or equal to 55 kg; or (iii) 9 mg once every two weeks to a pediatric patient with a body weight less than 30 kg. In some embodiments, the pediatric patient is 6 years of age or older.

A further exemplary weight-band dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)), after failure of at least two prior lines of systemic therapy, at a dosage of (i) 22 mg once every two weeks to an adult patient or a pediatric patient with a body weight greater than 55 kg; (ii) 14 mg once every two weeks to a pediatric patient with a body weight greater than or equal to about 30 kg and less than or equal to 55 kg; or (iii) 9 mg once every two weeks to a pediatric patient with a body weight less than 30 kg. In some embodiments, the pediatric patient is 6 years of age or older.

An exemplary weight-band dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO: 2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) at a dosage of (i) about 23 mg once every two weeks to a human subject with a body weight greater than about 60 kg; (ii) about 17 mg once every two weeks to a human subject with a body weight greater than or equal to about 40 kg and less than or equal to about 60 kg; or (iii) about 10.5 mg once every two weeks to a human subject with a body weight less than about 40 kg. In some embodiments, the human subject is an adult patient. In some embodiments, the human subject is a pediatric patient. In some embodiments, the pediatric patient is 6 years of age or older.

A further exemplary weight-band dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)), after failure of at least two prior lines of systemic therapy, at a dosage of (i) about 23 mg once every two weeks to a human subject with a body weight greater than about 60 kg; (ii) about 17 mg once every two weeks to a human subject with a body weight greater than or equal to about 40 kg and less than or equal to about 60 kg; or (iii) about 10.5 mg once every two weeks to a human subject with a body weight less than about 40 kg. In some embodiments, the human subject is an adult patient. In some embodiments, the human subject is a pediatric patient. In some embodiments, the pediatric patient is 6 years of age or older.

A further exemplary weight-band dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)) at a dosage of (i) 23 mg once every two weeks to a human subject with a body weight greater than 60 kg; (ii) 17 mg once every two weeks to a human subject with a body weight greater than or equal to 40 kg and less than or equal to 60 kg; or (iii) 10.5 mg once every two weeks to a human subject with a body weight less than 40 kg. In some embodiments, the human subject is an adult patient. In some embodiments, the human subject is a pediatric patient. In some embodiments, the pediatric patient is 6 years of age or older.

A further exemplary weight-band dosing regimen comprises intravenous administration of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)), after failure of at least two prior lines of systemic therapy, at a dosage of (i) 23 mg once every two weeks to a human subject with a body weight greater than 60 kg; (ii) 17 mg once every two weeks to a human subject with a body weight greater than or equal to 40 kg and less than or equal to 60 kg; or (iii) 10.5 mg once every two weeks to a human subject with a body weight less than 40 kg. In some embodiments, the human subject is an adult patient. In some embodiments, the human subject is a pediatric patient. In some embodiments, the pediatric patient is 6 years of age or older.

A dose of a pharmaceutical composition comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)) can be administered biweekly (every two weeks).

A pharmaceutical composition may include a therapeutically effective amount of an anti-CSF-1R antibody described herein (e.g., axatilimab or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO: 3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and/or a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)).

The term “therapeutically effective amount of an agent is that amount sufficient to effect beneficial or desired results, for example, clinical results, and, as such, a “therapeutically effective amount” depends upon the context in which it is being applied. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### Indications

Anti-CSF-1R antibodies described herein and pharmaceutical compositions described herein comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO: 3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO:6); and the VL CDR3 comprises the amino acid sequence LQDSEYPWT (SEQ ID NO:7)) can be used to treat chronic graft-versus-host disease (cGVHD) in a human subject in need thereof. In some embodiments, the human subject is an adult patient. In some embodiments, the human subject is a pediatric patient. In some embodiments, the pediatric patient is 6 years of age or older. In some embodiments, the human subject was prior treated with ibrutinib, ruxolitinib, or ibrutinib and ruxolitinib. In some embodiments, the human subject was prior treated with ibrutinib, ruxolitinib, belumosudil, or a combination of any of these. In some embodiments, the human subject exhibited manifestations of cGVHD in greater than or equal to four organs at baseline. In some embodiments, the greater than or equal to four organs at baseline are selected from mouth, esophagus, small intestine, colon, liver, joints and fascia, eyes, skin, and lungs. In some embodiments, the greater than or equal to four organs at baseline comprise the eyes, the skin, or the eyes and the skin. In some embodiments, the cGVHD is recurrent or refractory active cGVHD. In some embodiments, the cGVHD is severe cGVHD. In some embodiments, the cGVHD is recurrent or refractory active cGVHD after two or more lines of systemic therapy. In some embodiments, the cGVHD is recurrent or refractory active cGVHD after three or more lines of systemic therapy. In some embodiments, the cGVHD is recurrent or refractory active cGVHD after four or more lines of systemic therapy. In some embodiments, the cGVHD is recurrent or refractory active cGVHD after five or more lines of systemic therapy. In some embodiments, the cGVHD is recurrent or refractory active cGVHD after six or more lines of systemic therapy.

Another aspect comprises anti-CSF-1R antibodies described herein or pharmaceutical compositions described herein comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)) for use in the treatment of cGVHD in a human subject in need thereof. In some embodiments, the human subject is an adult patient. In some embodiments, the human subject is a pediatric patient. In some embodiments, the pediatric patient is 6 years of age or older. In some embodiments, the human subject was prior treated with ibrutinib, ruxolitinib, or ibrutinib and ruxolitinib. In some embodiments, the human subject was prior treated with ibrutinib, ruxolitinib, belumosudil, or a combination of any of these. In some embodiments, the human subject exhibited manifestations of cGVHD in greater than or equal to four organs at baseline. In some embodiments, the greater than or equal to four organs at baseline are selected from mouth, esophagus, small intestine, colon, liver, joints and fascia, eyes, skin, and lungs. In some embodiments, the greater than or equal to four organs at baseline comprise the eyes, the skin, or the eyes and the skin. In some embodiments, the cGVHD is recurrent or refractory active cGVHD. In some embodiments, the cGVHD is severe cGVHD. In some embodiments, the cGVHD is recurrent or refractory active cGVHD after two or more lines of systemic therapy. In some embodiments, the cGVHD is recurrent or refractory active cGVHD after three or more lines of systemic therapy. In some embodiments, the cGVHD is recurrent or refractory active cGVHD after four or more lines of systemic therapy. In some embodiments, the cGVHD is recurrent or refractory active cGVHD after five or more lines of systemic therapy. In some embodiments, the cGVHD is recurrent or refractory active cGVHD after six or more lines of systemic therapy.

Another aspect comprises anti-CSF-1R antibodies described herein or pharmaceutical compositions described herein comprising a mixture of axatilimab (or an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASSLQD (SEQ ID NO:12); and the VL CDR3 comprises the amino acid sequence LQDSEYPWT (SEQ ID NO:7)), and a sequence variant thereof (e.g., an anti-CSF-1R antibody comprising a VH domain comprising VH CDR1, VH CDR2, and VH CDR3, wherein the VH CDR1 comprises the amino acid sequence GFSLTTYGMGVG (SEQ ID NO:2); the VH CDR2 comprises the amino acid sequence NIWWDDDKYYNPSLKN (SEQ ID NO:3); and the VH CDR3 comprises the amino acid sequence IGPIKYPTAPYRYFDF (SEQ ID NO:4); and wherein the antibody comprises a VL domain comprising VL CDR1, VL CDR2, and VL CDR3, wherein the VL CDR1 comprises the amino acid sequence LASEDIYDNLA (SEQ ID NO:5); the VL CDR2 comprises the amino acid sequence YASYLQD (SEQ ID NO: 6); and the VL CDR3 comprises the amino acid sequence LQDSEYPWT (SEQ ID NO: 7)) in the manufacture of a medicament for treating cGVHD in a human subject in need thereof. In some embodiments, the human subject is an adult patient. In some embodiments, the human subject is a pediatric patient. In some embodiments, the pediatric patient is 6 years of age or older. In some embodiments, the human subject was prior treated with ibrutinib, ruxolitinib, or ibrutinib and ruxolitinib. In some embodiments, the human subject was prior treated with ibrutinib, ruxolitinib, belumosudil, or a combination of any of these. In some embodiments, the human subject exhibited manifestations of cGVHD in greater than or equal to four organs at baseline. In some embodiments, the greater than or equal to four organs at baseline are selected from mouth, esophagus, small intestine, colon, liver, joints and fascia, eyes, skin, and lungs. In some embodiments, the greater than or equal to four organs at baseline comprise the eyes, the skin, or the eyes and the skin. In some embodiments, the treated subject has advanced cGVHD. In some embodiments, the cGVHD is recurrent or refractory active cGVHD. In some embodiments, the cGVHD is severe cGVHD. In some embodiments, the cGVHD is recurrent or refractory active cGVHD after two or more lines of systemic therapy. In some embodiments, the cGVHD is recurrent or refractory active cGVHD after three or more lines of systemic therapy. In some embodiments, the cGVHD is recurrent or refractory active cGVHD after four or more lines of systemic therapy. In some embodiments, the cGVHD is recurrent or refractory active cGVHD after five or more lines of systemic therapy. In some embodiments, the cGVHD is recurrent or refractory active cGVHD after six or more lines of systemic therapy.

In some embodiments, the treated subject has received at least two previous cGVHD treatments. In some embodiments, the treated subject has received at least three previous cGVHD treatments. In some embodiments, the treated subject has received at least four previous cGVHD treatments. In some embodiments, the treated subject has received at least five previous cGVHD treatments. In some embodiments, the treated subject has received at least six previous cGVHD treatments. In some embodiments, the treated subject has received greater than four previous cGVHD treatments. In some embodiments, the treated subject has received between four and six previous cGVHD treatments. In some embodiments, the treated subject has received greater than six previous cGVHD treatments. In some embodiments, the human subject was prior treated with ibrutinib, ruxolitinib, or ibrutinib and ruxolitinib.

In some embodiments, the human subject exhibited manifestations of cGVHD in greater than or equal to four organs at baseline. In some embodiments, the greater than or equal to four organs at baseline are selected from mouth, esophagus, small intestine, colon, liver, joints and fascia, eyes, skin, and lungs. In some embodiments, the greater than or equal to four organs at baseline comprise the eyes, the skin, or the eyes and the skin.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Bioactivity and Efficacy of Axatilimab Sequence Variant

During the manufacture of axatilimab, in a cell line containing nucleic acids encoding the heavy and light chains of axatilimab, it was observed that a sequence variant (SV) of the antibody was repeatedly and unexpectedly produced. The SV is characterized by a serine 53 to tyrosine (S53Y) substitution, as compared to axatilimab, in the light chain CDR2 region and was found to be present in about 2.5% of total light chains produced. This change of an amino acid residue in a CDR of axatilimab has the potential to impact bioactivity and/or efficacy. Given that a change in an amino acid residue within a CDR of an antibody can affect antibody activity, the unexpected production of the SV during the manufacture of axatilimab had the potential to render unusable any axatilimab manufacturing process that results in the production of the SV. Therefore, a review of in vitro potency to compare antibody lots (with and without the SV) was performed to assess the impact of the SV.

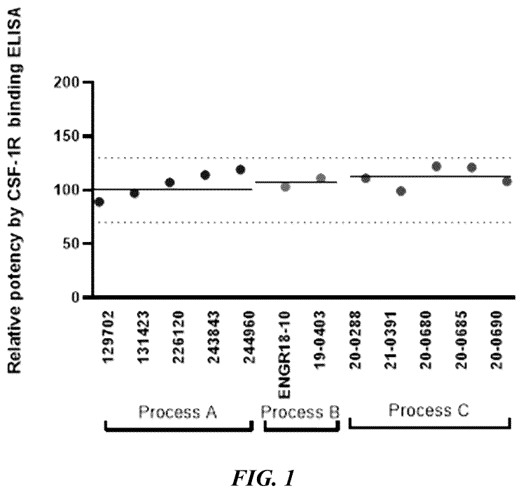

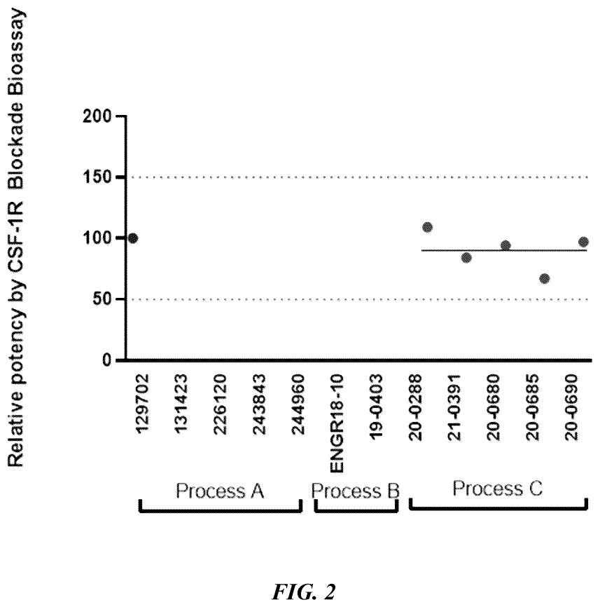

Comparable potency results in CSF-1R competitive binding ELISA (FIG. 1) were observed in lots containing ˜2.5% of the SV (and ˜97.5% of axatilimab) as compared to lots containing 100% axatilimab (i.e., no SV). In addition, the potency measured by the CSF-1R blockade bioassay (FIG. 2) also showed comparable results between lots containing ˜2.5% of the SV (and ˜97.5% of axatilimab) and lots containing 100% axatilimab. The presence of ˜2.5% S53Y SV thus showed no impact on bioactivity/efficacy based on these in vitro assays.

### Example 2: Structure/Function Characterization

To determine if the amount of SV present in a given lot affects potency, mixtures were generated containing the SV and axatilimab at varying percentages. The S53Y SV form was generated through an expression vector and partially purified by protein A chromatography. Axatilimab (control) was also generated by the same technique to minimize any potential changes in binding due to expression technique. The sequence of both the axatilimab control and the substitution of serine to tyrosine in the SV were confirmed by reduced intact, non-reduced intact LC-MS analysis, and reduced peptide mapping by LC-MS/MS analysis.

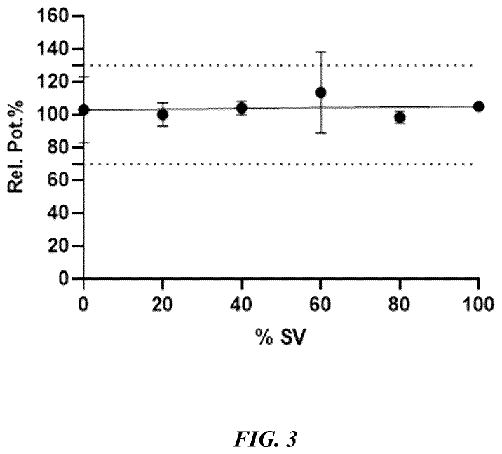

Potency of the mixtures was measured by competitive binding CSF-1R ELISA with the results shown in FIG. 3. The assessment of potency shown is relative to the interim reference, which contains 100% axatilimab (and 0% of the SV). Mixtures containing the S53Y SV remained fully potent, even at the 100% abundance level. The measured relative potency of all the tested samples remains consistent and between 70 and 130%, regardless of the abundance level of S53Y SV in the mixture ranging from 0 to 100%. No trend was observed, which was confirmed by statistical analysis via a linear regression (p-value of 0.77).

### Example 3: Pharmacokinetics/Pharmacodynamics (PK/PD)

The charge heterogeneity and FcRn binding of lots containing ˜2.5% of the SV (and ˜97.5% of axatilimab) to those containing only axatilimab (and no SV) were assessed. It was determined that the charge isoforms of lots containing the SV and lots containing only axatilimab (and no SV) were comparable. In addition to charge, Fc receptor binding kinetics of lots containing the SV and lots containing only axatilimab (and no SV) were evaluated. Comparable binding kinetics in FcRn, FcγRI, FcγRIIa (R and H variants), FcγRIIIa (F and V variants) and C1q were observed in lots containing the SV and lots containing only axatilimab (and no SV) (Table 1). Additionally, there was no change in FcRn binding kinetics, indicating the impact of the SV on PK/PD profile is low.

TABLE 1
Axatilimab Fc Receptors Binding Results

| | Non-SV-Containing Lot | |
|---|---|---|
| | (axatilimab only) | SV-Containing Lot |
| Protein | K_{D} (nM)^{a} | K_{D} (nM)^{a} |
| FcRn^{b} | 884 ± 29.0 | 848 ± 12.9 |
| FcγRI^{c} | 18.6 ± 0.53 | 18.4 ± 0.46 |
| FcγRIIa^{b} R Variant | 20,800 ± 150 | 21,200 ± 155 |
| FcγRIIa^{b} H Variant | 20,500 ± 200 | 19,800 ± 710 |
| FcγRIIb^{b} | 21,500 ± 300 | 22,400 ± 610 |
| FcγRIIIa^{b} F Variant | 41,900 ± 10,200 | 46,000 ± 10,200 |
| FcγRIIIa^{b} V Variant | 23,100 ± 760 | 21,600 ± 3,250 |
| FcγRIIIb^{b} NA1 Variant | N.A.^{d} | N.A.^{d} |
| FcγRIIIb^{b} NA2 Variant | N.A.^{d} | N.A.^{d} |
| C1q^{b} | 41.3 ± 3.04 | 41.7 ± 4.11 |

^{a}The data are averages ± standard deviations of 3 independent experiments.
^{b}kₐ and k_{d} were not determined due to the fast on-off nature of the binding.
^{c}K_{D} determined by k_{d}/kₐ. Measured kₐ (1.47 × 105, 1.51 × 105 and 1.44 × 105 (M⁻¹s⁻¹) and k_{d} (2.75 × 10⁻³, 2.72 × 10⁻³ and 2.73 × 10⁻³ (s⁻¹)) for Non-SV containing Lot. Measured kₐ (1.53 × 10⁵, 1.55 × 10⁵ and 1.47 × 10⁵ (M⁻¹s⁻¹)) and k_{d} (2.80 × 10⁻³, 2.79 × 10⁻³ and 2.78 × 10⁻³ (s⁻¹)) for SV containing Lot.
^{d}No significant binding observed.

### Example 4: Safety

In the AGAVE-201 clinical study, 19 patients with chronic graft-versus-host disease were treated with lots of axatilimab containing ˜2.5% of the SV (and ˜97.5% of axatilimab). Of the 19 patients, 8 were initially randomized to the 0.3 mg/kg Q2W dose, 7 to the 1 mg/kg Q2W dose, and 4 to the 3 mg/kg Q2W dose. Of the 8 patients in the 0.3 mg/kg Q2W dose cohort, prior to receiving a lot of axatilimab containing ˜2.5% of the SV, 2 patients escalated their dose to 1 mg/kg Q2W as allowed by the active protocol at that time. At the time of transition to the lot of axatilimab containing ˜2.5% of the SV, axatilimab dosing was as follows: 2 patients at 0.3 mg/kg Q2W, 6 patients at 0.6 mg/kg Q4W, 2 patients at 1 mg/kg Q2W, 7 patients at 2 mg/kg Q4W, and 2 patients at 3 mg/kg Q4W. The dose of axatilimab at the time of transition to lots of axatilimab containing ˜2.5% of the SV is summarized by initially randomized dose group in Table 2 below.

TABLE 2
Axatilimab Dose at the Time of Transition
to SV-Containing Drug Product

| | Dose at Transition to SV- |
|---|---|
| Initially Randomized Dose | Containing Drug Product |
| 0.3 mg/kg q2w (n = 8) | 0.3 mg/kg q2w (n = 2) |
| | 0.6 q4w (n = 5) |
| | 1 mg/kg q2w (n = 1) |
| 1 mg/kg q2w (n = 7) | 1 mg/kg q2w (n = 1) |
| | 2 mg/kg q4w (n = 6) |
| 3 mg/kg q4w (n = 4) | 3 mg/kg q4w (n = 2) |
| | 2 mg/kg q4w (n = 1) |
| | 0.6 mg/kg q4w (n = 1) |

Patients who transitioned to lots of axatilimab containing ˜2.5% of the SV received a median of 3 cycles (range: 1-8) of axatilimab after transition and before the 3-month safety DCO date. Out of 19 patients who transitioned to lots of axatilimab containing ˜2.5% of the SV, 5 have ended treatment during the 3-month safety follow-up window, with only one patient ending treatment due to progression of the disease. Patient-level review of available data did not show any negative impact of lots of axatilimab containing ˜2.5% of the SV on continued efficacy data. Therefore, further analyses focused on safety findings emerging after the transition to lots of axatilimab containing ˜2.5% of the SV and their comparison with the treatment emergent adverse event (TEAE) experience of transitioning patients before the institution of lots of axatilimab containing ˜2.5% of the SV. Analyses of TEAEs and adverse drug reactions (ADRs) identified no new or unexpected safety findings, with emerging profile largely unchanged and not impacted by use of lots of axatilimab containing ˜2.5% of the SV. In particular, no new infusion-related reactions were noted in these patients. A summary of ADRs before and after switching to lots of axatilimab containing ˜2.5% of the SV is presented in Table 3. There have been a small number of ADRs after switching to lots of axatilimab containing ˜2.5% of the SV, none of which can be directly attributed to the level of SV.

TABLE 3
Incidence of Adverse Effects Before and After Switch to SV Containing Drug Product

| | 0.3 mg/kg Q2W (n = 8) | | 1 mg/kg Q2W (n = 7) | | | 3 mg/kg Q4W (n = 4) | | |
|---|---|---|---|---|---|---|---|---|
| | Before SV | After SV | Before SV | | After SV | Before SV | | After SV |
| | Containing | Containing | Containing | | Containing | Containing | | Containing |
| | Drug | Drug | Drug | | Drug | Drug | | Drug |
| ADR Type, n (%) | Product | Product | Product | | Product | Product | | Product |
| Any ADR | 5 (62.5) | 1 (12.5) | 7 | (100) | 3 (42.9) | 4 | (100) | 1 (25.0) |
| ALT increased | 1 (12.5) | 0 | 1 | (14.3) | 0 | 3 | (75.0) | 0 |
| AST increased | 1 (12.5) | 0 | 2 | (28.6) | 0 | 4 | (100) | 0 |
| Blood AlkPh increased | 0 | 0 | 2 | (28.6) | 1 (14.3) | 2 | (50.0) | 0 |
| GGT increased | 0 | 0 | 1 | (14.3) | 0 | 3 | (75.0) | 0 |
| Infusion related reaction | 1 (12.5) | 0 | 2 | (28.6) | 0 | 0 | | 0 |
| Blood CPK increased | 2 (25.0) | 1 (12.5) | 3 | (42.9) | 1 (14.3) | 3 | (75.0) | 0 |
| Blood LDH increased | 2 (25.0) | 0 | 2 | (28.6) | 0 | 2 | (50.0) | 0 |
| Fatigue | 2 (25.0) | 0 | 4 | (57.1) | 1 (14.3) | 2 | (50.0) | 0 |
| Hypertension | 0 | 0 | 3 | (42.9) | 1 (14.3) | 0 | | 0 |
| Hypophosphatemia | 1 (12.5) | 1 (12.5) | 0 | | 0 | 1 | (25.0) | 0 |
| Lipase increased | 3 (37.5) | 0 | 1 | (14.3) | 0 | 2 | (50.0) | 1 (25.0) |
| Amylase increased | 0 | 0 | 0 | | 0 | 1 | (25.0) | 0 |
| Pruritus | 0 | 0 | 2 | (28.6) | 0 | 2 | (50.0) | 0 |

| | | | | | | | |
|---|---|---|---|---|---|---|---|
| Periorbital oedema | 0 | 0 | 0 | 0 | 1 | (25.0) | 0 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody that binds to human colony stimulating factor 1 receptor (CSF-1R), the antibody comprising a variable heavy (VH) domain comprising VH complementarity determining region (CDR)1 (VHCDR1), VH CDR2, and VH CDR3, wherein:
the VH CDR1 comprises the amino acid sequence GFSLTTYGMGVG (SEQ ID NO:2);
the VH CDR2 comprises the amino acid sequence NIWWDDDKYYNPSLKN (SEQ ID NO: 3); and
the VH CDR3 comprises the amino acid sequence IGPIKYPTAPYRYFDF (SEQ ID NO: 4); and
wherein the antibody comprises a variable light (VL) domain comprising VL CDR1, VL CDR2, and VL CDR3, wherein:
the VL CDR1 comprises the amino acid sequence LASEDIYDNLA (SEQ ID NO:5);
the VL CDR2 comprises the amino acid sequence YASYLQD (SEQ ID NO:6); and
the VL CDR3 comprises the amino acid sequence LQDSEYPWT (SEQ ID NO:7).

2. The antibody of claim 1, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:8).

3. The antibody of claim 1, wherein the VL domain comprises the amino acid sequence DIQMTQSPSSLSASVGDRVTITCLASEDIYDNLAWYQQKPGKAPKLLIYYASYLQDGVPS RFSGSGSGTDYTLTISSLQPEDFATYYCLQDSEYPWTFGGGTKVEIK (SEQ ID NO:9).

4. The antibody of claim 1, wherein the VH domain comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:8), and wherein the VL domain comprises the amino acid sequence DIQMTQSPSSLSASVGDRVTITCLASEDIYDNLAWYQQKPGKAPKLLIYYASYLQDGVPS RFSGSGSGTDYTLTISSLQPEDFATYYCLQDSEYPWTFGGGTKVEIK (SEQ ID NO:9).

5. The antibody of claim 1, wherein the antibody comprises a heavy chain and a light chain, and wherein the heavy chain comprises the amino acid sequence set forth in SEQ ID NO: 10 and the light chain comprises the amino acid sequence set forth in SEQ ID NO:11.

6. The antibody of claim 1, wherein the antibody comprises a heavy chain and a light chain, and wherein the heavy chain comprises the amino acid sequence set forth in SEQ ID NO: 15 and the light chain comprises the amino acid sequence set forth in SEQ ID NO:11.

7. A nucleic acid sequence or nucleic acid sequences encoding the heavy and/or light chain(s) of the antibody of claim 1.

8. An expression vector comprising the nucleic acid sequence or nucleic acid sequences of claim 7.

9. A host cell comprising the expression vector of claim 8.

10. A pharmaceutical composition comprising the antibody of claim 1 and at least one pharmaceutically acceptable diluent, carrier or excipient.

11. A pharmaceutical composition comprising a first antibody, a second antibody, and at least one pharmaceutically acceptable diluent, carrier or excipient, wherein the first antibody is the antibody of claim 1, and wherein the second antibody is an antibody that binds to human colony stimulating factor 1 receptor (CSF-1R) comprises a variable heavy (VH) domain comprising VH complementarity determining region (CDR)1 (VHCDR1), VH CDR2, and VH CDR3, wherein:
the VH CDR1 comprises the amino acid sequence GFSLTTYGMGVG (SEQ ID NO:2);
the VH CDR2 comprises the amino acid sequence NIWWDDDKYYNPSLKN (SEQ ID NO: 3); and
the VH CDR3 comprises the amino acid sequence IGPIKYPTAPYRYFDF (SEQ ID NO: 4); and
wherein the antibody comprises a variable light (VL) domain comprising VL CDR1, VL CDR2, and VL CDR3, wherein:
the VL CDR1 comprises the amino acid sequence LASEDIYDNLA (SEQ ID NO:5);
the VL CDR2 comprises the amino acid sequence YASSLQD (SEQ ID NO:12); and
the VL CDR3 comprises the amino acid sequence LQDSEYPWT (SEQ ID NO:7).

12. The pharmaceutical composition of claim 11, wherein the VH domain of the second antibody comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:8), and wherein the VL domain of the second antibody comprises DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:13).

13. The pharmaceutical composition of claim 11, wherein the second antibody comprises a heavy chain and a light chain, and wherein the heavy chain of the second antibody comprises the amino acid sequence set forth in SEQ ID NO: 10 and the light chain the second antibody comprises the amino acid sequence set forth in SEQ ID NO:14.

14. The pharmaceutical composition of claim 11, wherein the second antibody comprises a heavy chain and a light chain, and wherein the heavy chain of the second antibody comprises the amino acid sequence set forth in SEQ ID NO:15 and the light chain the second antibody comprises the amino acid sequence set forth in SEQ ID NO:14.

15. The pharmaceutical composition of claim 11, wherein the VH domain of the first antibody comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:8), wherein the VL domain of the first antibody comprises DIQMTQSPSSLSASVGDRVTITCLASEDIYDNLAWYQQKPGKAPKLLIYYASYLQDGVPS RFSGSGSGTDYTLTISSLQPEDFATYYCLQDSEYPWTFGGGTKVEIK (SEQ ID NO:9), wherein the VH domain of the second antibody comprises the amino acid sequence EVTLKESGPALVKPTQTLTLTCTFSGFSLTTYGMGVGWIRQPPGKALEWLANIWWDDD KYYNPSLKNRLTISKDTSKNQVVLTMTNMDPVDTATYYCARIGPIKYPTAPYRYFDFW GQGTMVTVS (SEQ ID NO:8), and wherein the VL domain of the second antibody comprises DIQMTQSPSSLSASVGDRVTITCLASEDIYDNLAWYQQKPGKAPKLLIYYASSLQDGVPS RFSGSGSGTDYTLTISSLQPEDFATYYCLQDSEYPWTFGGGTKVEIK (SEQ ID NO:13).

16. The pharmaceutical composition of claim 11, wherein:
the first antibody comprises a heavy chain and a light chain, wherein the heavy chain of the first antibody comprises the amino acid sequence set forth in SEQ ID NO:10 and the light chain the first antibody comprises the amino acid sequence set forth in SEQ ID NO:11; and
the second antibody comprises a heavy chain and a light chain, wherein the heavy chain of the second antibody comprises the amino acid sequence set forth in SEQ ID NO: 10 and the light chain the second antibody comprises the amino acid sequence set forth in SEQ ID NO:14.

17. The pharmaceutical composition of claim 11, wherein:
the first antibody comprises a heavy chain and a light chain, wherein the heavy chain of the first antibody comprises the amino acid sequence set forth in SEQ ID NO:15 and the light chain the first antibody comprises the amino acid sequence set forth in SEQ ID NO:11; and
the second antibody comprises a heavy chain and a light chain, wherein the heavy chain of the second antibody comprises the amino acid sequence set forth in SEQ ID NO: 15 and the light chain the second antibody comprises the amino acid sequence set forth in SEQ ID NO:14.

18. The pharmaceutical composition of claim 11, wherein the first antibody is present in an amount less than about 5.0%, less than about 4.5%, less than about 4.0%, less than about 3.5%, less than about 3.0%, less than about 2.5%, less than about 2.0%, less than about 1.5%, less than about 1.0%, or less than about 0.5% of the total amount of antibody in the pharmaceutical composition.

19. The pharmaceutical composition of claim 11, wherein the first antibody is present in an amount of 0.5% to 5.0% of the total amount of antibody in the pharmaceutical composition.

20. The pharmaceutical composition of claim 11, wherein the first antibody is present in an amount of 2.0% to 3.0% of the total amount of antibody in the pharmaceutical composition.

21. The pharmaceutical composition of claim 11, wherein the first antibody is present in an amount of about 2.5% of the total amount of antibody in the pharmaceutical composition.

22. The pharmaceutical composition of claim 11, wherein the first antibody is present in an amount of 0.5% to 5.0% of the total amount of antibody in the pharmaceutical composition, and wherein the second antibody is present in an amount of 95.0% to 99.5% of the total amount of antibody in the pharmaceutical composition.

23. The pharmaceutical composition of claim 11, wherein the first antibody is present in an amount of 2.0% to 3.0% of the total amount of antibody in the pharmaceutical composition, and wherein the second antibody is present in an amount of 97.0% to 98.0% of the total amount of antibody in the pharmaceutical composition.

24. The pharmaceutical composition of claim 11, wherein the first antibody is present in an amount of about 2.5% of the total amount of antibody in the pharmaceutical composition, and wherein the second antibody is present in an amount of about 97.5% of the total amount of antibody in the pharmaceutical composition.

25. A method of treating chronic graft-versus-host disease (cGVHD) in a human subject in need thereof, the method comprising administering to the human subject a therapeutically effective amount of the antibody of claim 1.

26. The method of claim 25, wherein the antibody is administered intravenously once every two weeks at a total antibody dose of 0.3 mg per kg of body weight of the human subject.

27. The method of claim 25, wherein:
(i) the human subject is an adult patient or a pediatric patient with a body weight greater than about 55 kg and the antibody or pharmaceutical composition is administered intravenously once every two weeks at a total antibody dose of about 22 mg;
(ii) the human subject is a pediatric patient with a body weight greater than or equal to about 30 kg and less than or equal to about 55 kg and the antibody or pharmaceutical composition is administered intravenously once every two weeks at a total antibody dose of about 14 mg; or
(iii) the human subject is a pediatric patient with a body weight less than about 30 kg and the antibody or pharmaceutical composition is administered intravenously once every two weeks at a total antibody dose of about 9 mg.

28. The method of claim 25, wherein:
(i) the human subject has a body weight greater than about 60 kg and the antibody or pharmaceutical composition is administered intravenously once every two weeks at a total antibody dose of about 23 mg;
(ii) the human subject has a body weight greater than or equal to about 40 kg and less than or equal to about 60 kg and the antibody or pharmaceutical composition is administered intravenously once every two weeks at a total antibody dose of about 17 mg; or
(iii) the human subject has a body weight less than about 40 kg and the antibody or pharmaceutical composition is administered intravenously once every two weeks at a total antibody dose of about 10.5 mg.

29. The method of claim 25, wherein the antibody or pharmaceutical composition is administered after failure of at least two prior lines of systemic therapy.

30. A method of treating chronic graft-versus-host disease (cGVHD) in a human subject in need thereof, the method comprising administering to the human subject a therapeutically effective amount of the pharmaceutical composition of claim 11.


SEQUENCE LISTING (15 sequences)

SEQ ID NO: 1 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 2 - protein, 12 aa, synthetic construct
GFSLTTYGMGVG

SEQ ID NO: 3 - protein, 16 aa, synthetic construct
NIWWDDDKYYNPSLKN

SEQ ID NO: 4 - protein, 16 aa, synthetic construct
IGPIKYPTAPYRYFDF

SEQ ID NO: 5 - protein, 11 aa, synthetic construct
LASEDIYDNLA

SEQ ID NO: 6 - protein, 7 aa, synthetic construct
YASYLQD

SEQ ID NO: 7 - protein, 9 aa, synthetic construct
LQDSEYPWT

SEQ ID NO: 8 - protein, 125 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVS

SEQ ID NO: 9 - protein, 107 aa, synthetic construct
DIQMTQSPSSLSASVGDRVTITCLASEDIYDNLAWYQQKPGKAPKLLIYYASYLQDGVPSRFSGSGSGTDYTLTISSLQPEDFATYYCLQDSEYPWTFGGGTKVEIK

SEQ ID NO: 10 - protein, 453 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, synthetic construct
DIQMTQSPSSLSASVGDRVTITCLASEDIYDNLAWYQQKPGKAPKLLIYYASYLQDGVPSRFSGSGSGTDYTLTISSLQPEDFATYYCLQDSEYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 7 aa, synthetic construct
YASSLQD

SEQ ID NO: 13 - protein, 107 aa, synthetic construct
DIQMTQSPSSLSASVGDRVTITCLASEDIYDNLAWYQQKPGKAPKLLIYYASSLQDGVPSRFSGSGSGTDYTLTISSLQPEDFATYYCLQDSEYPWTFGGGTKVEIK

SEQ ID NO: 14 - protein, 214 aa, synthetic construct
DIQMTQSPSSLSASVGDRVTITCLASEDIYDNLAWYQQKPGKAPKLLIYYASSLQDGVPSRFSGSGSGTDYTLTISSLQPEDFATYYCLQDSEYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 452 aa, synthetic construct
EVTLKESGPALVKPTQTLTLTCTFSGFSLTTYGMGVGWIRQPPGKALEWLANIWWDDDKYYNPSLKNRLTISKDTSKNQVVLTMTNMDPVDTATYYCARIGPIKYPTAPYRYFDFWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG